(19) United States
(12) Patent Application Publication
(10) Pub. No.: US 2022/0031912 A1
(43) Pub. Date: Feb. 3, 2022

(54) QUALITY OF A CELLULAR GRAFT

(71) Applicant: Sybleu, Inc., Cheyenne, WY (US)

(72) Inventor: David Koos, La Mesa, CA (US)

(21) Appl. No.: 17/376,084

(22) Filed: Jul. 14, 2021

Related U.S. Application Data
(60) Provisional application No. 63/051,789, filed on Jul. 14, 2020.

(51) Int. Cl. A61L 27/36; C12N 13/00; C12N 5/0789

(52) U.S. Cl. CPC A61L 27/3691; A61L 27/3616; A61L 27/3641; A61L 27/3687; C12N 2501/22; C12N 5/0647; A61L 2430/28; C12N 2529/10; C12N 13/00

(57) ABSTRACT

Disclosed are methods, devices, and techniques useful for enhancing function of an organ or cellular graft through photoceutical manipulation. In one embodiment a hematopoietic graft is treated with one or more wavelengths of low level laser irradiation at a sufficient energy to enhance homing and engraftment. In another embodiment the recipient long bones are treated with one or more wavelengths of low level laser irradiation at a sufficient energy to enhance chemoattraction and growth factor secretion on recipient stromal cells. Application of the invention includes areas of cellular transplants such as islet and hepatic cell grafts.

### FIELD OF THE INVENTION

The invention pertains generally to the area of cellular transplantation, more specifically, the invention relates to the need for enhancing engraftment and function of cellular grafts after transplantation. The invention also belongs to the field of photoceuticals and utilization of light therapy for augmentation of graft efficiency.

### CROSS-REFERENCE TO RELATED APPLICATIONS

This application claims priority to U.S. Provisional Application No. 63/051,789, titled “Improving Quality of a Cellular Graft”, and filed Jul. 14, 2020, which is hereby incorporated by reference in its entirety.

### BACKGROUND

Cellular transplantation offers the possibility of curing many diseases previously considered uncurable. Perhaps the biggest success of this approach is witnessed in the area of hematopoietic stem cell transplants, which have cured not only patients with leukemias, but also several genetic abnormalities (1). In situations of hematopoietic stem cell transplantation recipient bone marrow is usually ablated in order to provide “space” for the donor bone marrow cells. Additionally, when treatment of hematological malignancies is being performed, the recipient cells need to be eradicated in order to cure the neoplasia. Unfortunately the process of hematological ablation involves exposing the patient to a period of immune deficiency. During this period multiple infections occur, which in a non-insignificant number of cases are lethal. Accordingly, it is of great interest in the art to accelerate the process of donor bone marrow engraftment and hematopoietic reconstitution. Another cause of stem cell transplant associated mortality is graft versus host disease (GVHD). In this condition the donor stem cell graft contains immune cells that initiate attack on the recipient. Cord blood transplants have significantly less GVHD as compared to bone marrow, however cord blood cells have a delayed engraftment time, thus exposing the patient to elevated risk of infections. Thus there is a need in the art to enhance stem cell engraftment and reconstitution in cells such as cord blood stem cells.

Cellular transplants have been useful in the treatment of type I diabetes. The Edmonton Protocol is an example of islet transplantation that has demonstrated positive results (2). Unfortunately 2-3 donor pancreases are needed per recipient and long-term graft survival rarely occurs. The efficacy of the Edmonton Protocol could be increased if the problems of poor vascularization, post-implantation apoptosis, and growth factor secretion of the implanted islets could be modified. Improved cell graft quality has been achieved with methods such as co-addition of bone marrow cells (3), treatment with hyperbaric oxygen (4), or addition of growth factors (5). Co-administration of bone marrow cells with the islet graft may cause potential problems in terms of ectopic tissue growth. Furthermore this procedure has not been performed clinically on a wide-spread basis. Addition of growth factors may trigger subdormant tumors. Therefore there are currently limited methods to increase viability of a cellular graft. This problem is not only apparent in the case of islet transplantation but also for neuronal and hepatocyte transplantation.

### SUMMARY

Various aspects of the invention are directed to methods of increasing efficacy of a cell for use in transplantation by pretreatment of said cell prior to transplantation with a laser irradiation of at least one wavelength, said wavelength(s) in a range between about 400 nanometers and about 1070 nanometers administered for a total energy of 100·mu·W/cm·sup·2 to approximately 10 W/cm·sup 0.2.

Preferred methods are directed to embodiments wherein said transplanted cell is selected from a hematopoietic stem cell, an islet cell population, a hepatocyte population, or a neural cell population.

Preferred methods are directed to embodiments wherein said hematopoietic stem cell is capable of reconstituting the hematopoietic system of an animal.

Preferred methods are directed to embodiments wherein said hematopoietic stem cell is obtained from a source selected from: a) bone marrow; b) mobilized peripheral blood; c) cord blood; and d) fetal liver.

Preferred methods are directed to embodiments wherein said mobilization of peripheral blood is performed by administration of a sufficient quantity of an agent selected from a group comprising of: a) G-CSF; b) GM-CSF; c) Mozibil; and d) cyclophosphamide.

Preferred methods are directed to embodiments wherein said bone marrow hematopoietic cell consists of a heterogeneous population of mononuclear cells.

Preferred methods are directed to embodiments wherein said bone marrow hematopoietic stem cell consists of isolated CD34 cells.

Preferred methods are directed to embodiments wherein said bone marrow hematopoietic stem cell consists of isolated CD133 cells.

Preferred methods are directed to embodiments wherein said mobilized peripheral blood stem cells are comprised of a heterogeneous leukopheresis product extracted from a donor after mobilization.

Preferred methods are directed to embodiments wherein said mobilized peripheral blood stem cells are comprised of an isolated CD34 cell derived from heterogeneous leukopheresis product extracted from a donor after mobilization.

Preferred methods are directed to embodiments wherein said cord blood derived stem cells comprise a heterogeneous population of cord blood mononuclear cells.

Preferred methods are directed to embodiments wherein said cord blood derived stem cells comprise a population selected for expression of CD34.

Preferred methods are directed to embodiments wherein said cord blood derived stem cells comprise a population selected for expression of CD133.

Preferred methods are directed to embodiments wherein said fetal liver derived stem cells comprise a heterogeneous population of cord blood mononuclear cells.

Preferred methods are directed to embodiments wherein said fetal liver derived stem cells comprise a population of cord blood mononuclear cells selected for expression of the marker CD34.

Preferred methods are directed to embodiments wherein said fetal liver derived stem cells comprise a population of cord blood mononuclear cells selected for expression of the marker CD133.

Preferred methods are directed to embodiments wherein said cell population is autologous or allogeneic to the recipient.

Preferred methods are directed to embodiments wherein said islet population is harvested from a donor pancreas by infusing the pancreatic ducts with ET-Kyoto solution or equivalent thereto.

Preferred methods are directed to embodiments wherein a stimulator of hematopoietic stem cell activity is combined with said wavelength(s) in a range between about 400 nanometers and about 1070 nanometers administered for a total energy of 100 ·mu·W/cm·sup·2 to approximately 10 W/cm·sup.2.

Preferred methods are directed to embodiments wherein said stimulator is selected from a group comprising of: G-CSF, GM-CSF, thrombopoietic, flt-3L, IL-1, IL-6, IL-3, and IL-11.

Preferred embodiments include a method of augmenting efficacy of a cellular graft subsequent to transplantation by treating the area in which said graft will be transplanted with laser irradiation of at least one wavelength, said wavelength(s) in a range between about 400 nanometers and about 1070 nanometers administered for a total energy of 100 ·mu·W/cm·sup·2 to approximately 10 W/cm·sup·2.

Preferred methods are directed to embodiments wherein said area of transplantation is the liver in the case of hepatocytes and islets.

Preferred methods are directed to embodiments wherein said islet transplantation is performed according to the Edmonton Protocol.

Preferred methods are directed to embodiments wherein said area of transplantation is the bone marrow in the case of hematopoietic stem cell grafts.

Preferred methods are directed to embodiments wherein energy and wavelength for intervention is chosen based on ability to stimulate production of a chemoattractant molecule from said area in which graft will be transplanted.

Preferred methods are directed to embodiments wherein said chemoattractant molecule is selected from a group comprising of: a) cytokines; b) chemokines; and c) peptides.

Preferred methods are directed to embodiments wherein said chemoattract is stromal derived factor-1.

Preferred embodiments are directed to methods of decreasing neutropenic time in a patient subsequent to a hematopoietic cell transplantation consisting of: a) extraction of a hematopoietic stem cell source; b) administration of low level laser irradiation to said hematopoietic stem cell source at a sufficient energy and wavelength to increase hematopoietic reconstitution activity of said hematopoietic stem cell source; c) administration of treated cells; and/or d) providing low level laser irradiation to long bones of said patient at a sufficient energy and wavelength to increase hematopoietic stem cell engraftment.

Preferred methods are directed to embodiments wherein low level laser irradiation may be administrated by laser or light emitting diode.

Preferred embodiments are directed to methods of treating a cytopenia consisting of: a) extraction of a hematopoietic stem cell source; b) administration of low level laser irradiation to said hematopoietic stem cell source at a sufficient energy and wavelength to increase hematopoietic reconstitution activity of said hematopoietic stem cell source; c) administration of treated cells; and/or d) providing low level laser irradiation to long bones of said patient at a sufficient energy and wavelength to increase hematopoietic stem cell engraftment.

Preferred methods are directed to embodiments wherein said cytopenia is the result of an insult to the hematopoietic system.

Preferred methods are directed to embodiments wherein said cytopenia is selected from a group comprising of: a) thrombocytopenia; b) neutropenia; c) anemia; and d) lymphopenia.

Preferred methods are directed to embodiments wherein said hematopoietic insult is the result of chemotherapy.

Preferred methods are directed to embodiments wherein said hematopoietic insult is the result of radiation.

Preferred embodiment are directed to methods of treating a cytopenia consisting of administration of low level laser irradiation to the long bones of a patient in need at a wavelength and power sufficient to modify hematopoietic stem cell activity.

Preferred methods are directed to embodiments wherein said modification of hematopoietic stem cell activity is proliferation.

Preferred methods are directed to embodiments wherein said modification of hematopoietic stem cell activity is differentiation.

### BRIEF DESCRIPTION OF THE DRAWINGS

FIG. 1 is a depiction of a medical device for stimulation of cellular grafts.

### DETAILED DESCRIPTION OF THE INVENTION

The invention provides means of “preactivating” a cellular graft before implantation. Said “preactivation” refers to induction of biochemical processes within the graft so as to allow for: a) increased viability; b) augmented function; c) accelerated integration with the tissue in which implantation of cellular graft has occurred. In the context of hematopoietic stem cell grafts the present invention provides methods of augmenting ability of transplanted stem cells to enter and incorporate into the bone marrow niche, to self renew and proliferate once established in said niche, and to generate blood cells.

In one embodiment the invention teaches the use of low level laser irradiation as a means of increasing practivating stem cells before infusion. Wavelengths and energy of low level laser irradiation useful for stimulation of stem cell proliferation may be chosen based on mitogenesis, colony forming unit, cytokine stimulation or SCID-repopulating unit experiments. Stem cell populations include bone marrow, cord blood, or mobilized peripheral blood mononuclear cells, as well as selected CD34 and/or CD133 cells. It is known to one of skill in the art that wavelengths and energies useful for the practice of the invention may be chosen based ability to stimulate proliferation, cytokine secretion or activity of cells similar to hematopoietic stem cells. In one particular embodiment stem cells are treated for approximately 60 seconds with a helium neon (He—Ne) laser at a wavelength of 632.8 nm and at a power of approximately 2.8 mW following guidance provided in previous published work in the area of porcine granulosa cells (6). Alternatively, the same wavelength may be used at an energy of approximately 1.5 J/cm² which was demonstrated to stimulate cytokine production from cultured keratinocyte cells (7). Other wavelengths may be used based on guidance provided from publications describing stimulatory activity on non-hematopoietic stem cells. For example, administration of 0.96 J/cm² of energy at 635 nm was shown sufficient to increase mesenchymal stem cells homing to infarct areas (8). This wavelength and energy is applied in the context of the present invention as a means of pre-activating hematopoietic stem cell prior to implantation. Similar wavelength at 0.96 J/cm² was capable of eliciting cytokine production and augmenting differentiation processes (9), thus could be used within the context of the current invention. Additionally, wavelengths of 810 nm have also been useful in stimulating mesenchymal stem cell homing and activity at energies between 1-3 J/cm (10, 11) and are also contemplated within the current invention for hematopoietic stem cell pre-activation. The cited publications did not have the intention of activating stem cells for use in the particularly unique process of preactivation of hematopoietic stem cells before infusion but rather demonstrate that light-based intervention is capable of eliciting cellular activities. The unique process of the current invention is the new application of the well-known activities of low level lasers to the process of hematopoietic reconstitution.

Within the context of the current invention low level laser irradiation may be used in combination with other agents known to augment various processes of hematopoietic reconstitution. Means of accelerating this process include modification of progenitor cells by alteration of adhesion molecule activity through such manipulations such as fucosylation of cell surface molecules, cytokine pretreatment, exposure to heat, or treatment with epigenetic acting factors such as 5-azacytidine, valproic acid, trichostatin-A, or sodium phenylbutyrate.

In the practice of the invention stem cells useful for hematopoietic reconstitution may be preactivated and subsequently administered together with growth factors. Administration of said growth factors may occur prior to stem cell administration, concurrently with, or subsequently after. Granulocyte colony stimulating factor (G-CSF) is an example of a growth factor used in clinical practice to augment hematopoietic, and particularly granulocytic differentiation. In one embodiment of the current invention G-CSF is used as an adjuvant to accelerate hematopoietic reconstitution of low level laser pretreated hematopoietic stem cells.

One of the major dose-limiting toxicities of chemotherapy is cytopenia. Use of G-CSF to augment neutrophil production after or concurrent with chemotherapy is common-place in oncology clinics. The current invention teaches that administration of low level laser irradiation to the long bones of a hematopoietically-compromised patient induces acceleration of hematopoietic recovery and shortens cytopenic time. Low level irradiation frequency and power must be sufficient to penetrate the long bones and may be chosen between about 400 nanometers to about 1070 nanometers administered for a total energy of 100 ·mu·W/cm·sup·2 to approximately 10 W/cm·sup·2. In one particular embodiment a wavelength of approximately 808 nm is used.

Administration of low level laser irradiation to long bones may also be used for acceleration of stem cell reconstitution after administration. In this embodiment wavelengths are chosen based on ability to increase production of the chemokine stromal derived factor (SDF)-1 from the bone marrow stromal cells. Wavelengths useful for this application range from 400 nanometers to about 1070 nanometers administered for a total energy of 100 ·mu·W/cm·sup·2 to approximately 10 W/cm·sup·2. One particular approach for ensuring tissue penetration is using similar parameters as reported by the Photothera group in which 808 nm wavelength was sufficient to penetrate the skull bone and elicit effects in patients post-stroke (12). The current invention teaches that screening for optimized wavelengths may be performed in vitro using mesenchymal stem cells or osteoblasts as target cells and assessing production of SDF-1 using methods such as enzyme linked immunosorbent assay (ELISA) or reverse transcriptase polymerase chain reaction (RT-PCR).

The preactivation of stem cells before administration in myeloablated patients may be performed with a medical device comprised of a closed system. Said device contains an inlet that connects in a closed manner to a cryobag using connection systems available in the art such as Leuer Lock. Access to cells pre and post-irradiation is provided using valves that allow for sample collection. This is an important part of quality control for the cells. Classical parameters of interest include cell viability and morphology.

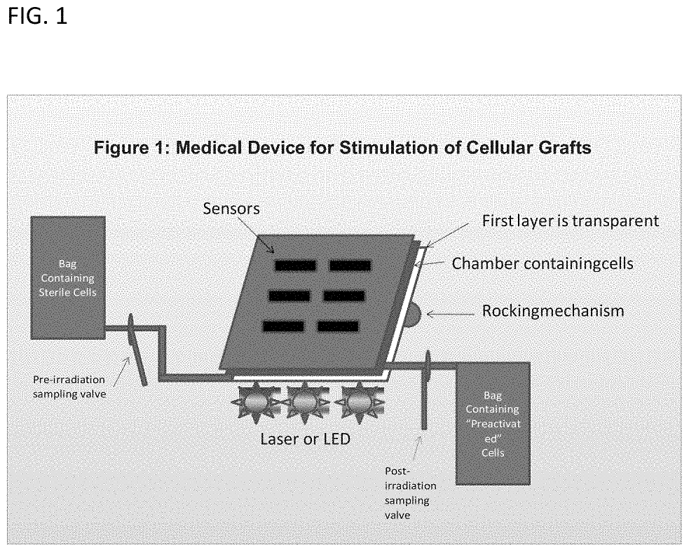

Said device further consists of a surface area of sufficient size so that cells from the cryobag or other sterile source can be exposed to irradiation in a uniform or semi-uniform manner. Said device may further possess a rocking mechanism to ensure cell dispersion. Under the surface area a low level laser is provided that administers the desired frequency and intensity of laser irradiation to said cells (FIG. 1). Said device may be useful for other cellular grafts besides hematopoietic cells. For example, the cells used may be islets for treatment of diabetes. In the case of islets parameters of preactivation may be different than ones used for hematopoietic stem cells. In the case of islets important considerations include the induction of the anti-apoptotic gene bcl-2, stimulation of angiogenic factors such as VEGF and IGF-1, as well as stimulation of proliferation. One of skill in the art will use the teachings of the current invention to screen for wavelengths and energy densities capable of accomplishing these goals.

### REFERENCES

Gratwohl, A., and Baldomero, H. 2009. Trends of hematopoietic stem cell transplantation in the third millennium. Curr Opin Hematol 16:420-426.
Ryan, E. A., Lakey, J. R., Rajotte, R. V., Korbutt, G. S., Kin, T., Imes, S., Rabinovitch, A., Elliott, J. F., Bigam, D., Kneteman, N. M., et al. 2001. Clinical outcomes and insulin secretion after islet transplantation with the Edmonton protocol. Diabetes 50:710-719.
Sakata, N., Chan, N. K., Chrisler, J., Obenaus, A., and Hathout, E. Bone marrow cells produce nerve growth factor and promote angiogenesis around transplanted islets. World J Gastroenterol 16:1215-1220.
Sakata, N., Chan, N. K., Ostrowski, R. P., Chrisler, J., Hayes, P., Kim, S., Obenaus, A., Zhang, J. H., and Hathout, E. Hyperbaric oxygen therapy improves early posttransplant islet function. Pediatr Diabetes.
Park, K. S., Kim, Y. S., Kim, J. H., Choi, B., Kim, S. H., Tan, A. H., Lee, M. S., Lee, M. K., Kwon, C. H., Joh, J. W., et al. Trophic molecules derived from human mesenchymal stem cells enhance survival, function, and angiogenesis of isolated islets after transplantation. Transplantation 89:509-517.
Gregoraszczuk, E., Dobrowolski, J. W., and Galas, J. 1983. Effect of low intensity laser beam on steroid dehydrogenase activity and steroid hormone production in cultured porcine granulosa cells. Folia Histochem Cytochem (Krakow) 21:87-92.
Yu, H. S., Chang, K. L., Yu, C. L., Chen, J. W., and Chen, G. S. 1996. Low-energy helium-neon laser irradiation stimulates interleukin-1 alpha and interleukin-8 release from cultured human keratinocytes. J Invest Dermatol 107:593-596.
Zhang, H., Hou, J. F., Shen, Y., Wang, W., Wei, Y. J., and Hu, S. 2009. Low Level Laser Irradiation Precondition to Create Friendly Milieu of Infarcted Myocardium and Enhance Early Survival of Transplanted Bone Marrow Cells. J Cell Mol Med.
Hou, J. F., Zhang, H., Yuan, X., Li, J., Wei, Y. J., and Hu, S. S. 2008. In vitro effects of low-level laser irradiation for bone marrow mesenchymal stem cells: proliferation, growth factors secretion and myogenic differentiation. Lasers Surg Med 40:726-733.
Tuby, H., Maltz, L., and Oron, U. 2007. Low-level laser irradiation (LLLI) promotes proliferation of mesenchymal and cardiac stem cells in culture. Lasers Surg Med 39:373-378.
Tuby, H., Maltz, L., and Oron, U. 2009. Implantation of low-level laser irradiated mesenchymal stem cells into the infarcted rat heart is associated with reduction in infarct size and enhanced angiogenesis. Photomed Laser Surg 27:227-233.
Zivin, J. A., Albers, G. W., Bornstein, N., Chippendale, T., Dahlof, B., Devlin, T., Fisher, M., Hacke, W., Holt, W., Ilic, S., et al. 2009. Effectiveness and safety of transcranial laser therapy for acute ischemic stroke. Stroke 40:1359-1

1. A method of increasing efficacy of cells for use in transplantation by pretreatment of said cells prior to transplantation with laser irradiation of at least one wavelength, said wavelength(s) in a range between about 400 nanometers and about 1070 nanometers administered for a total energy of 100 μW/cm to approximately 10 W/cm cm; and transplanting said irradiated cells to a patient in need.

2. The method of claim 1, wherein said transplanted cells are selected from the group consisting of; hematopoietic stem cell population, an islet cell population, a hepatocyte population, and a neural cell population.

3. (canceled)

4. The method of claim 2, wherein said hematopoietic stem cell population is obtained from a source selected from the group consisting of: a) bone marrow; b) mobilized peripheral blood; c) cord blood; and d) fetal liver.

5. (canceled)

6. The method of claim 4, wherein said bone marrow hematopoietic cell population comprises a heterogeneous population of mononuclear cells.

7. (canceled)

8. (canceled)

9. (canceled)

10. The method of claim 4, wherein said mobilized peripheral blood stem cells comprise an isolated CD34 cell population derived from a heterogeneous leukopheresis product extracted from a donor after mobilization.

11. (canceled)

12. (canceled)

13. (canceled)

14. (canceled)

15. (canceled)

16. (canceled)

17. (canceled)

18. (canceled)

19. (canceled)

20. The method of claim 1, wherein a stimulator selected from the group consisting of: G-CSF, GM-CSF, thrombopoietic, flt-3L, IL-1, IL-6, IL-3, and IL-11 is also administered to the cells prior to transplantation.

21. (canceled)

22. The method of claim 1, wherein said area of transplantation in the patient is the liver and the cells are selected from the group consisting of hepatocytes and islets.

23. The method of claim 22, wherein said transplantation is an islet transplantation, and is performed according to the Edmonton Protocol.

24. The method of claim 1, wherein said area of transplantation in the patient is the bone marrow and the cells are hematopoietic stem cells.

25. The method of claim 21, wherein energy and wavelength for intervention is chosen based on ability to stimulate production of a chemoattractant molecule from said area in which graft will be transplanted, and said chemoattractant molecule is selected from the group consisting of: a) cytokines; b) chemokines; and c) peptides.

26. (canceled)

27. The method of claim 25, wherein said chemoattractant is stromal derived factor-1.

28. The method of claim 2, further comprising providing low level laser irradiation to a long bone of said patient at a sufficient energy and wavelength to increase hematopoietic stem cell engraftment at the site of said long bone.

29. (canceled)

30. The method of claim 1, wherein the patient is suffering from cytopenia and said transplantation is performed in an amount sufficient to treat said cytopenia.

31. The method of claim 30, wherein said cytopenia is the result of an insult to the hematopoietic system.

32. The method of claim 31, wherein said cytopenia is selected from the group comprising of: a) thrombocytopenia; b) neutropenia; c) anemia; and d) lymphopenia.

33. The method of claim 31, wherein said hematopoietic insult is the result of chemotherapy.

34. The method of claim 31, wherein said hematopoietic insult is the result of radiation.

35. (canceled)

36. (canceled)

37. (canceled)
